(19) United States
(12) Patent Application Publication
(10) Pub. No.: US 2023/0416848 A1
(43) Pub. Date: Dec. 28, 2023

(54) SYSTEMS AND METHODS FOR ADVANCING REACTIONS BETWEEN MULTIPLE CHAMBERS OF A TESTING DEVICE

(71) Applicant: PRESIDENT AND FELLOWS OF HARVARD COLLEGE, CAMBRIDGE, MA (US)

(72) Inventors: Thomas E. Schaus, Cambridge, MA (US); Peng Yin, Cambridge, MA (US)

(21) Appl. No.: 18/035,619

(22) PCT Filed: Nov. 12, 2021
(86) PCT No.: PCT/US2021/059207

§ 371 Date: May 5, 2023

Related U.S. Application Data
(60) Provisional application No. 63/112,751, filed on Nov. 12, 2020.
(60) Provisional application No. 63/124,919, filed on Dec. 14, 2020.
(60) Provisional application No. 63/126,701, filed on Dec. 17, 2020.
(60) Provisional application No. 63/191,205, filed on May 20, 2021.
(60) Provisional application No. 63/270,350, filed on Oct. 21, 2021.

(51) Int. Cl. C12Q 1/70; C12Q 1/6844; B01L 3/00; B01L 7/00

(52) U.S. Cl. CPC C12Q 1/701; C12Q 1/6844; B01L 3/502715; B01L 7/52; B01L 2200/16; B01L 2200/0621; B01L 2200/0631; B01L 2300/0832; B01L 2300/123; B01L 2400/0478; B01L 2300/18

(57) ABSTRACT

A testing device includes an elongated member and a tube assembly. The tube assembly has a first end and a second end. The tube assembly is configured to receive the elongated member at the second end. The tube assembly includes a plurality of chambers, including a first chamber and a second chamber. The first chamber and the second chamber are separated by a membrane. The tube assembly further includes a spring positioned at the second end of the tube assembly. The tube assembly further includes a spring retainer configured to prevent the spring from decompressing when in a locked position and permit the spring to decompress when in an unlocked position.

### CROSS-REFERENCE TO RELATED APPLICATIONS

This application claims priority to and the benefits of U.S. Provisional Patent Application No. 63/112,751, filed on Nov. 12, 2020, U.S. Provisional Patent Application No. 63/124,919, filed on Dec. 14, 2020, U.S. Provisional Patent Application No. 63/126,701, filed on Dec. 17, 2020, U.S. Provisional Patent Application No. 63/191,205, filed on May 20, 2021, and U.S. Provisional Patent Application No. 63/270,350, filed on Oct. 21, 2021, each of which are hereby incorporated herein by reference in their entireties.

### STATEMENT REGARDING FEDERALLY SPONSORED RESEARCH

This invention was made with government support under GM133052 awarded by the National Institutes of Health. The government has certain rights in the invention.

### TECHNICAL FIELD

The present disclosure relates generally to devices and methods for performing tests on samples. Specifically, the present disclosure is directed to a testing device with multiple chambers that advances at least a portion of an elongated member between a first chamber and a second chamber of the testing device.

### BACKGROUND

Pandemics or epidemics that require mass testing (e.g., the COVID-19 pandemic) place strains on testing resources based on a centralized testing infrastructure, such as where a few testing labs or centers are involved in providing test results to the population at large. Democratizing or decentralizing of testing can greatly enhance efficiency by removing any bottlenecks associated with a centralized infrastructure. For example, test results can be provided much quicker to the population in a more decentralized testing environment. However, such decentralized testing requires materials and equipment that can be used by the public at large who may not be familiar with the testing process. Accordingly, the present disclosure is related to error-proofing testing equipment for various applications.

### SUMMARY

According to some implementations of the present disclosure, a device for testing is provided. The device includes an elongated member and a tube assembly. The tube assembly has a first end and a second end. The tube assembly is configured to receive the elongated member at the second end. The tube assembly includes a plurality of chambers, including a first chamber and a second chamber. The first chamber and the second chamber are separated by a membrane. The tube assembly further includes a spring positioned at the second end of the tube assembly. The tube assembly further includes a spring retainer configured to prevent the spring from decompressing when in a locked position and permit the spring to decompress when in an unlocked position.

According to some implementations of the present disclosure, a method for conducting chemical reactions is provided. The method includes (a) inserting one end of an elongated member into a second end of a tube assembly such that the one end of the elongated member extends into a first chamber of the tube assembly; (b) decompressing a spring positioned at the second end of the tube assembly by unlocking a spring retainer of the tube assembly; and (c) puncturing a membrane separating the first chamber of the tube assembly from a second chamber of the tube assembly such that the one end of the elongated member extends into the second chamber of the tube assembly.

According to some implementations of the present disclosure, a method for conducting chemical reactions is provided. The method includes (a) inserting a swab in a first chamber of a testing device, the first chamber containing a first fluid mixture; (b) decompressing a first spring positioned in a first spring chamber of the testing device, the first spring decompression causing the fluid mixture in the first chamber to flow into the first spring chamber of the testing device, the fluid mixture being filtered by silica en route to the first spring chamber; and (c) decompressing a second spring positioned in a second spring chamber of the testing device, the second spring decompression causing a second fluid within the second spring chamber of the testing device to be filtered by the silica en route to a second chamber.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

This patent or application file contains at least one drawing executed in color. Copies of this patent or patent application publication with color drawing(s) will be provided by the Office upon request and payment of the necessary fee;

FIG. 1 illustrates an exemplary testing device, according to some implementations of the present disclosure;

FIG. 2 illustrates an exploded view of the testing device of FIG. 1, according to some implementations of the present disclosure;

FIG. 3 illustrates a cross-section of the exploded view of the testing device of FIG. 1, according to some implementations of the present disclosure;

FIG. 4 illustrates a cross-section of the testing device of FIG. 1 in a first configuration, according to some implementations of the present disclosure;

FIG. 5 illustrates a cross-section of the testing device of FIG. 1 in a second configuration, according to some implementations of the present disclosure;

FIG. 6-1 illustrates a cross-section of a second exemplary testing device, according to some implementations of the present disclosure;

FIG. 6-2 illustrates a cross-section of a third exemplary testing device, according to some implementations of the present disclosure;

FIG. 6-3 illustrates a cross-section of a fourth exemplary testing device, according to some implementations of the present disclosure;

FIG. 6-4 illustrates a cross-section of a fifth exemplary testing device, according to some implementations of the present disclosure;

FIG. 7 illustrates a process for conducting a test, according to some implementations of the present disclosure;

FIG. 8A illustrates a cross-section of a first exemplary electronic device, according to some implementations of the present disclosure;

FIG. 8B illustrates a cross-section of a second exemplary electronic device, according to some implementations of the present disclosure;

FIG. 9 illustrates an exemplary modular assembly of electronic devices, according to some implementations of the present disclosure;

FIG. 10 illustrates a sixth exemplary testing device, according to some implementations of the present disclosure;

FIG. 11 illustrates a cross-section of the sixth testing device of FIG. 10, according to some implementations of the present disclosure;

FIG. 12A illustrates a cross-section of a seventh exemplary testing device, according to some implementations of the present disclosure;

FIG. 12B illustrates a cross-section of the seventh testing device in a first position, according to some implementations of the present disclosure;

FIG. 12C illustrates a cross-section of the seventh testing device in a second position, according to some implementations of the present disclosure;

FIG. 12D illustrates a cross-section of the seventh testing device in a third position, according to some implementations of the present disclosure;

FIG. 12E illustrates a cross-section of the seventh exemplary testing device in a fourth position, according to some implementations of the present disclosure;

FIG. 13 illustrates a cross-section of an eighth exemplary testing device, according to some implementations of the present disclosure;

FIG. 13-1 illustrates a cross-section of part of a ninth exemplary testing device, according to some implementations of the present disclosure;

FIG. 14 illustrates a cross-section of a tenth exemplary testing device, according to some implementations of the present disclosure; and

FIG. 15 illustrates steps for using an exemplary testing device, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Embodiments of the present disclosure provide a simple, inexpensive system and method of advancing biochemical reactions that require multiple sequential compartments or chambers, whether due to different reagents, temperatures, or other requirements. In some implementations, a mechanism for storing potential energy is described, which can be released by a simple, mechanical triggering device to effect a fluid movement between chambers. For example, the potential energy can be stored as a user- or pre-compressed spring, with a solenoid triggering the release of the spring energy and forcing the displacement of fluid from a first chamber to a second chamber by a syringe mechanism.

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

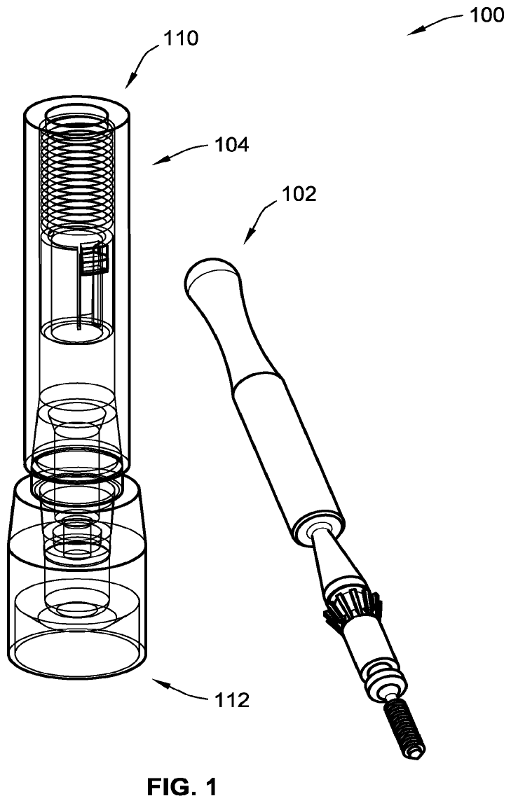

FIG. 1 illustrates a testing device 100, according to some implementations of the present disclosure. The testing device 100 can allow advancing reactions from one chamber of the testing device to another chamber of the testing device. The testing device 100 can be an inexpensive, disposable device. The testing device 100 can include an elongated member 102 and a tube assembly 104 with a first end 112 and a second end 110. The elongated member 102 can be a swab, a syringe, etc. The elongated member 102 is configured to be inserted in the tube assembly 104 at the second end 110. In FIG. 1, the tube assembly 104 is shown as a pre-assembled part of the testing device 100.

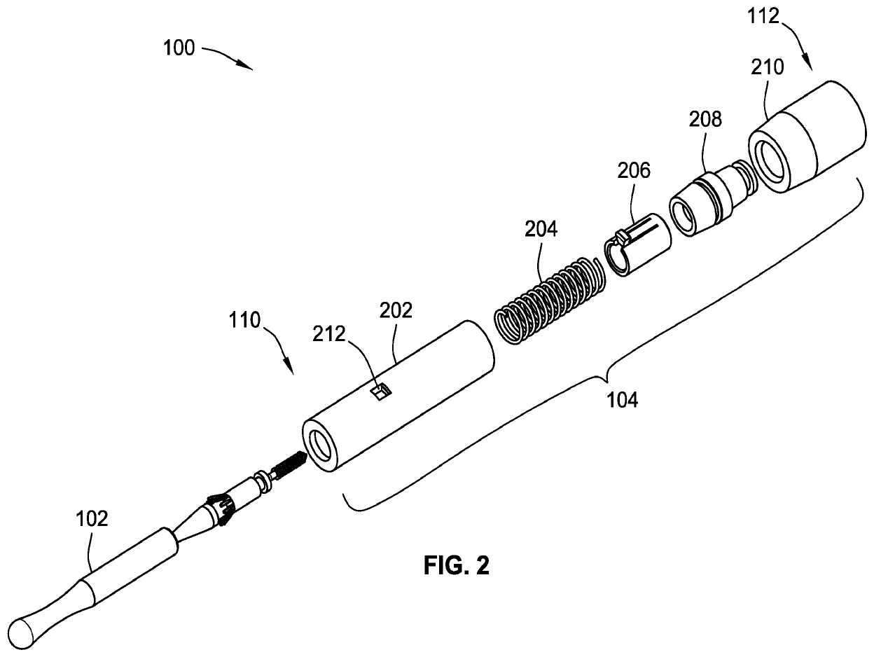

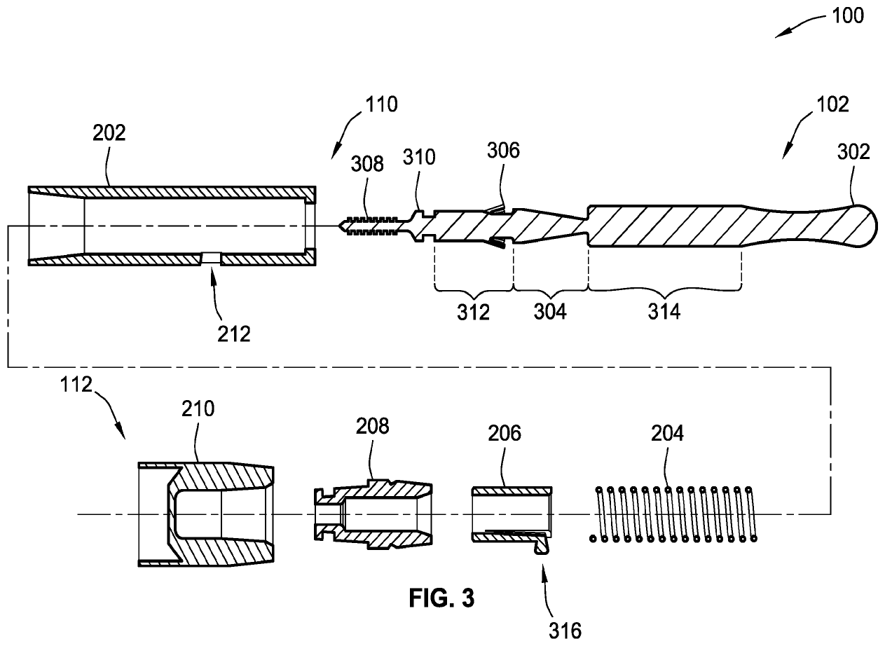

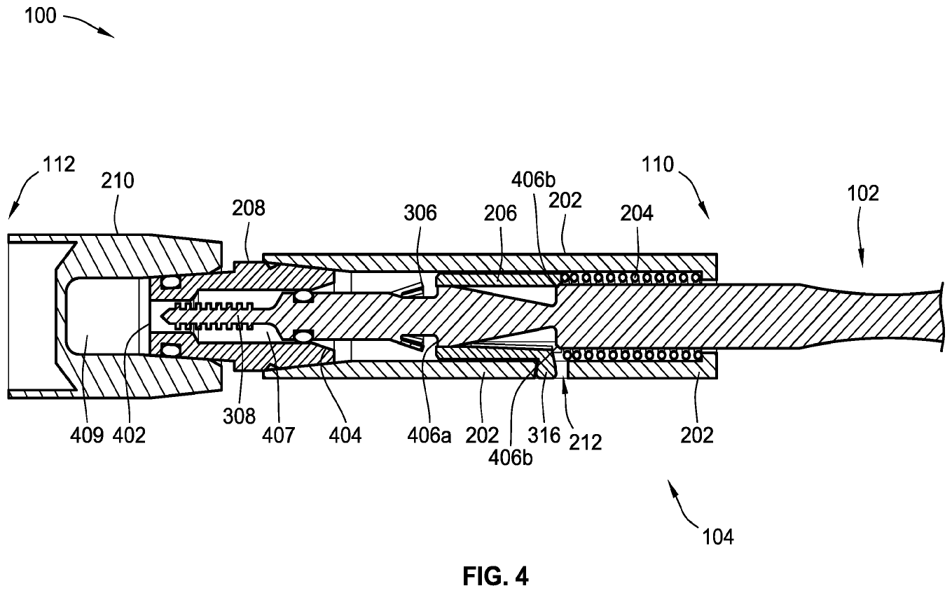

FIG. 2 illustrates an exploded view of components of the testing device 100, according to some implementations of the present disclosure. Specifically, FIG. 2 illustrates different parts of the pre-assembled tube assembly 104 of FIG. 1. The tube assembly 104 includes a first cylindrical member 202, a spring 204, a spring retainer 206, a first chamber casing 208 defining a first chamber 407 (FIG. 4), and a second chamber casing 210 defining a second chamber 409 (FIG. 4). FIG. 3 illustrates a cross-section of the exploded view of the testing device 100, according to some implementations of the present disclosure. Each of the elongated member 102, first cylindrical member 202, the spring retainer 206, the first chamber casing 208, and the second chamber casing 210 can be made of plastic or other materials. Examples of materials include acrylonitrile butadiene styrene (ABS), polypropylene, polycarbonate, nylon, 3D printed materials, etc. In some implementations components that are under stress or suffer mechanical creep (e.g., the spring retainer 206) can be metal. Examples of metals include steel, aluminum, etc. The spring 204, in some implementations, is a typically a steel spring.

The first cylindrical member 202 is a hollow structure and can include a key opening 212 along a sidewall of the first cylindrical member 202. The key opening 212 can be a hole in the first cylindrical member 202. An axis of the key opening 212 can be orthogonal to a longitudinal axis of the first cylindrical member 202. Although a single key opening 212 is depicted for the tube assembly 104, in some implementations, multiple key openings can be provided in succession along the sidewall of, for example, the first cylindrical member 202. The key opening 212 provides a catch that can receive a protrusion 316 or other portion of the spring retainer 206 when the tube assembly 104 is pre-assembled as depicted in FIG. 1. The spring retainer 206 can be hollow and substantially cylindrical, having an internal radius that is less than an internal radius of the first cylindrical member 202 of the tube assembly 104. The spring retainer 206 can fit within the hollow structure of the first cylindrical member 202.

When the protrusion 316 of the spring retainer 206 is not penetrating the key opening 212 of the first cylindrical member 202, the spring retainer 206 is unlocked and free to move within the hollow first cylindrical member 202. The spring 204 can push the spring retainer 206, causing the spring retainer 206 to move along the longitudinal axis of the first cylindrical member 202. When the protrusion 316 of the spring retainer 206 is penetrating the key opening 212 of the first cylindrical member 202, the spring retainer 206 is locked and thus maintains its position relative to the first cylindrical member 202.

The spring 204 is unable to cause the spring retainer 206 to move when the spring retainer 206 is in the locked position. In some implementations, the spring 204 is compressed, storing potential energy, when the spring retainer 206 is in the locked position. That is, the spring retainer 206 prevents the spring 204 from decompressing when in the locked position and permits the spring 204 to decompress and move the spring retainer 206 longitudinally, along the first cylindrical member 202, when in the unlocked position.

The spring retainer 206 being in a locked position, so as to not move along the tube assembly 104, or the spring retainer 206 being in an unlocked position, so as to allow movement along the tube assembly 104, can influence position of the elongated member 102. The elongated member 102 is inserted at the second end 110 of the tube assembly 104. The elongated member 102 can have a tip end 308 and a handle end 302. The tip end 308 of the elongated member 102 is inserted into the second end 110 of the tube assembly 104. The handle end 302 facilitates a user manually handling the elongated member 102 to position the elongated member 102 within the tube assembly 104.

The elongated member 102 can be configured to include multiple sections between the tip end 308 and the handle end 302. In some implementations, the elongated member 102 includes a cylindrical section 314 having a constant radius, a flared section 304 having a varying radius that increases over the length of the flared section 304 as it extends away from the cylindrical section 314, and a stopper section 312 having one or more flaps 306 for preventing the elongated member 102 from dislodging from the tube assembly 104 once inserted.

In some implementations, the elongated member 102 further includes a ridge 310. The ridge 310 can be used to secure an O-ring to the elongated member 102 so that when the tip end 308 of the elongated member 102 is inserted into the second end 110 of the tube assembly 104, the O-ring can form a close fit between the first chamber casing 208 and the elongated member 102. In some implementations, the O-ring can be substituted with components that have an easier manufacturing process (e.g., a ‘2-shot’ process using thermoplastic elastomers (TPEs) or similar) as an integrated seal. In some implementations, the elongated member 102 is configured to closely fit the first chamber casing 208, such that very little liquid is lost when the liquid is injected into the first chamber.

FIG. 4 illustrates a cross-section of the testing device 100 in a first configuration, according to some implementations of the present disclosure. The tube assembly 104 is pre-assembled in FIG. 4. In constructing the tube assembly 104, the first cylindrical member 202 is attached to the first chamber casing 208 which is attached to the second chamber casing 210. The spring retainer 206 is in the locked position such that the protrusion 316 of the spring retainer 206 penetrates the key opening 212 of the first cylindrical member 202. The spring 204 is compressed and positioned at the second end 110 of the tube assembly 104. The compressed spring 204 pushes against the spring retainer 206, but the protrusion 316 prevents the spring retainer 206 from moving away from the second end 110 of the tube assembly 104.

In the first configuration, the elongated member 102 is inserted in the tube assembly 104 such that the tip end 308 of the elongated member 102 is positioned within the first chamber 407 defined by the first chamber casing 208. A seal 402 or membrane separating a volume of the first chamber 407 defined by the first chamber casing 208 and a volume of the second chamber 409 defined by the second chamber casing 210 is provided. In the first configuration, the seal 402 is not broken by the tip end 308 of the elongated member 102. In some implementations, a seal can be provided on the first chamber casing 208 such that the tip end 308 of the elongated member 102 punctures the seal to enter the first chamber 407.

In the first configuration, the elongated member 102 is prevented from being further pushed into the tube assembly 104 by the spring retainer 206. A radius of the elongated member 102 at a portion 406a of the elongated member 102 is comparable to or greater than the inner radius of the spring retainer 206 such that pushing the elongated member 102 into the tube assembly 104 causes an interference between the elongated member 102 and the spring retainer 206 at the portion 406a, such that elongate member 102 can never pass further with respect to the spring retainer 206. The interference is provided here as an example, but other mechanical alternatives can be used to couple the elongated member 102 to the spring retainer 206. In some implementations, instead of the portion 406a of the elongated member 102, a portion 406b of the elongated member 102 can be provided with a radius that is comparable to or greater than the inner radius of the spring retainer 206, such that pushing the elongated member 102 into the tube assembly 104 causes an interference preventing the elongated member 102 from being pushed further with respect to the spring retainer 206. In some implementations, both of the portions 406a and 406b are provided on the elongated member 102. Other alternatives to causing an interference preventing the elongated member 102 from being pushed further with respect to the spring retainer 206 can be used as well. For example, instead of changing the radius along the elongated member 102 to cause the interference, the inner radius of the spring retainer 206 can be reduced at one end such that the portion 406a of the elongated member 102 is greater than the reduced inner radius at the one end.

While the interference between the elongated member 102 and the spring retainer 206 prevents the tip end 308 of the elongated member 102 from moving further toward the first end 112 of the tube assembly 104, the flap 306 prevents removal of the elongated member 102 from the tube assembly 104. The flap 306 is configured to become caught on the spring retainer 206 when trying to remove the elongated member 102 from the tube assembly 104. This serves to both prevent removal of the elongated member 102, but also to drive the elongated member 102 forward with the spring retainer 206. The particular implementation of the flap(s) 306 can be made in any number of ways, including the 3D printed configurations shown, a 2-component injection moldable assembly that is separated at or near the flaps (for the purpose of manufacturing, or movement of flap mechanism to within 206 to elicit the same one-way catch). The first chamber casing 208 includes a flared portion 404 that allows the flap 306 to move further down the cylindrical member 202 when the spring retainer 206 is released and placed in the unlocked position.

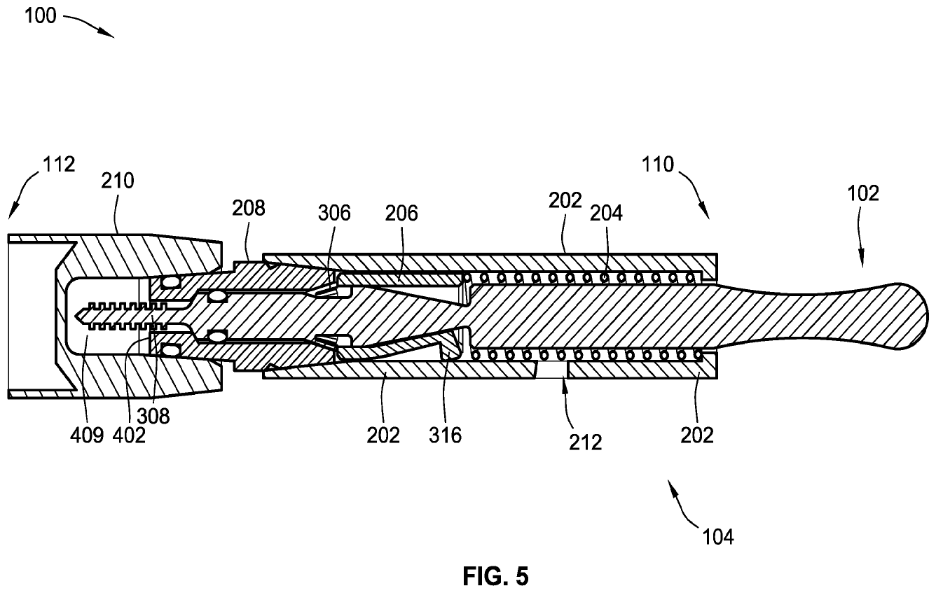

FIG. 5 illustrates a cross-section of the testing device 100 in a second configuration, according to some implementations of the present disclosure. In the second configuration, the spring retainer 206 is in the unlocked position such that the protrusion 316 no longer penetrates the key opening 212. The spring 204 is thus able to push the spring retainer 206 along the longitudinal axis of the tube assembly 104 toward the first end 112 of the tube assembly 104. Since the spring retainer 206 is coupled to the elongated member 102 at the portion 406a (FIG. 4) of the elongated member 102, the elongated member 102 is displaced a similar distance as the spring retainer 206, along the longitudinal axis of the tube assembly 104. In some implementations, due to manufacturing tolerance, the elongated member 102 can dislodge from the portion 406a, but the flap 306 engages with the spring retainer 206 and causes the elongated member 102 to be displaced the similar distance as the spring retainer 206, along the longitudinal axis of the tube assembly 104. In some implementations, the portion 406b (FIG. 4) of the elongated member 102 has a radius greater than the inner radius of the spring retainer 206, and the portion 406b engages with the spring retainer 206 when the elongated member 102 is displaced the similar distance as the spring retainer 206.

Displacing the elongated member 102 along the longitudinal axis of the tube assembly 104 toward the first end 112 of the tube assembly 104 results in the tip end 308 of the elongated member 102 puncturing the seal 402 that separates the volume of the first chamber 407 (FIG. 4) and the volume of the second chamber 409. In some implementations, puncturing the seal 402 to combine the volumes of both the first and second chambers causes reagents to mix between both chambers. In some implementations, a solenoid is used to push the protrusion 316 of the spring retainer 206 to dislodge the protrusion 316 from the key opening 212 causing the transition from the first configuration depicted in FIG. 4 to the second configuration depicted in FIG. 5. The spring retainer 206 can be deformed when the protrusion 316 is pushed such that at least a section of the spring retainer 206 containing the protrusion 316 has a deformed cross-section when compared to a section of the spring retainer 206 snug fit with the elongated member 102.

In some implementations, the testing device 100 is a disposable, multi-chambered device used only once to prevent cross-contamination. The solenoid that triggers transition from the first configuration to the second configuration can be part of a re-usable device, controlled by an electronic circuit such that timing of reaction advancement (and other features such as heating, or fluorimetry) are controlled. Although the spring 204 is provided as being within the tube assembly 104, in some implementations, the spring 204 can merely be a mechanism for providing mechanical energy for controllably advancing the elongated member 102 along the tube assembly 104. That is, the spring 204 can be part of the re-usable device that houses the solenoid. For example, the spring 204 can include the use of air pressure or compressed gas, or other mechanisms of potential energy formation, or simple electromagnets.

Although the first configuration and the second configuration are depicted in FIGS. 4 and 5, respectively, some implementations of the present disclosure include a testing device with more than two chambers. Such a testing device can be stacked to move fluids between more than the provided two chambers, e.g., from chamber 1 to chamber 2 by a first trigger, then from chamber 2 to chamber 3 by a second trigger, then from chamber 3 to chamber 4 by a third trigger, and so on. The multiple chambers can be separated by seals (e.g., the seal 402). In some implementations, the first cylindrical member 202 can be provisioned with multiple key openings (e.g., the key opening 212) such that the spring retainer 206 transitions between unlocking and locking into successive key openings towards the first end 112 of the tube assembly 104 after each successive trigger.

In some implementations, each successive chamber can include a reagent different from an adjacent chamber. For example, the first chamber can include a first reagent and the second chamber can include a second reagent. The first reagent can be different from the second reagent. Similarly, the second chamber and the third chamber can have different reagents such that the second reagent in the second chamber is different from a third reagent in the third chamber. In some implementations, the third reagent is the same as the first reagent. In some implementations, instead of having different reagents, each chamber is kept at a different temperature. In some implementations, different combinations of temperatures can be used in different chambers.

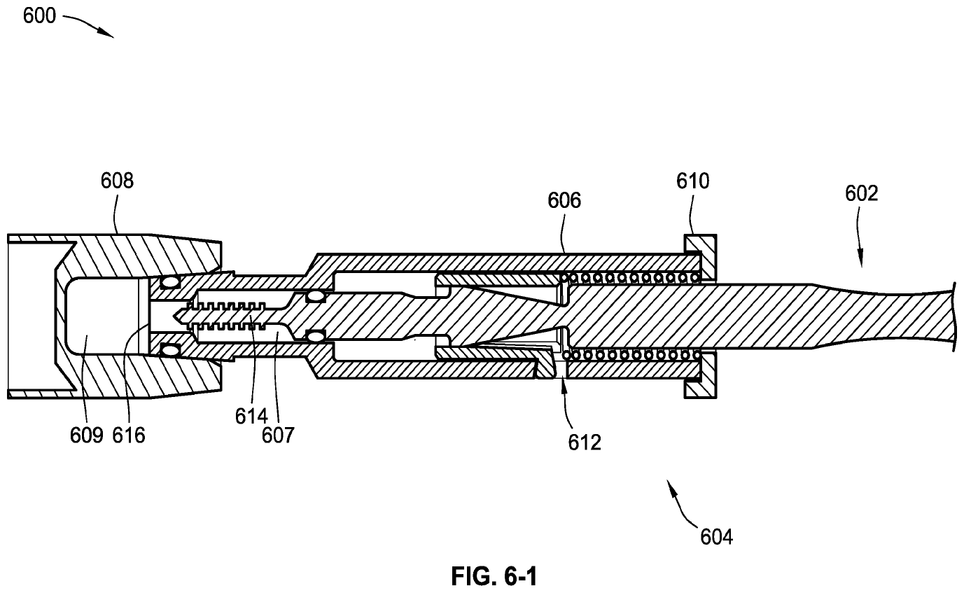

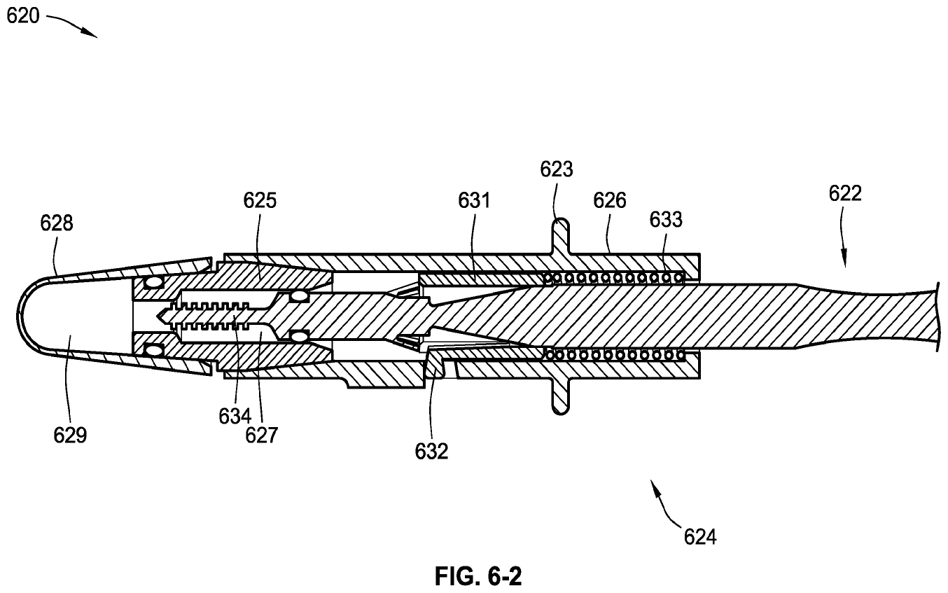

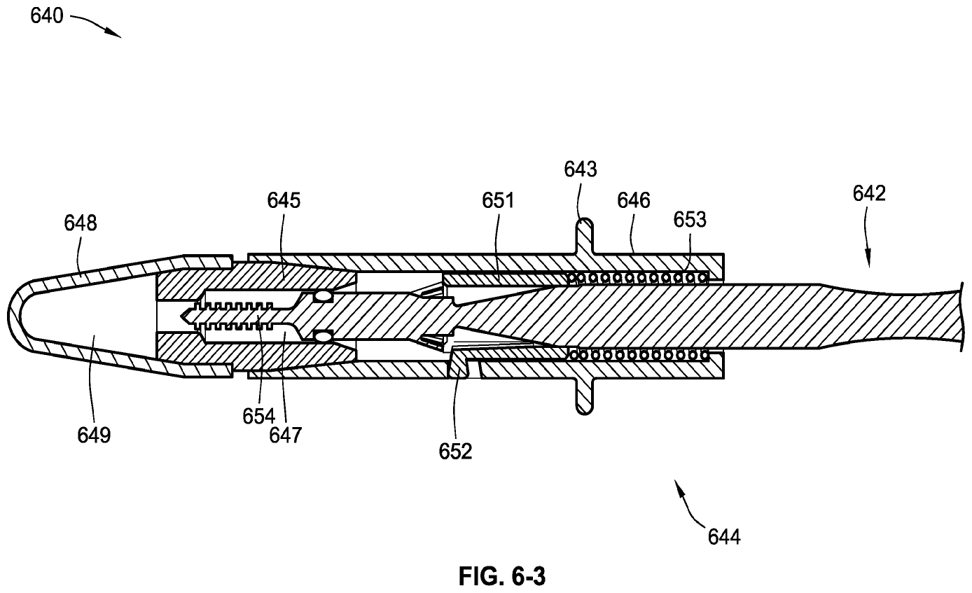

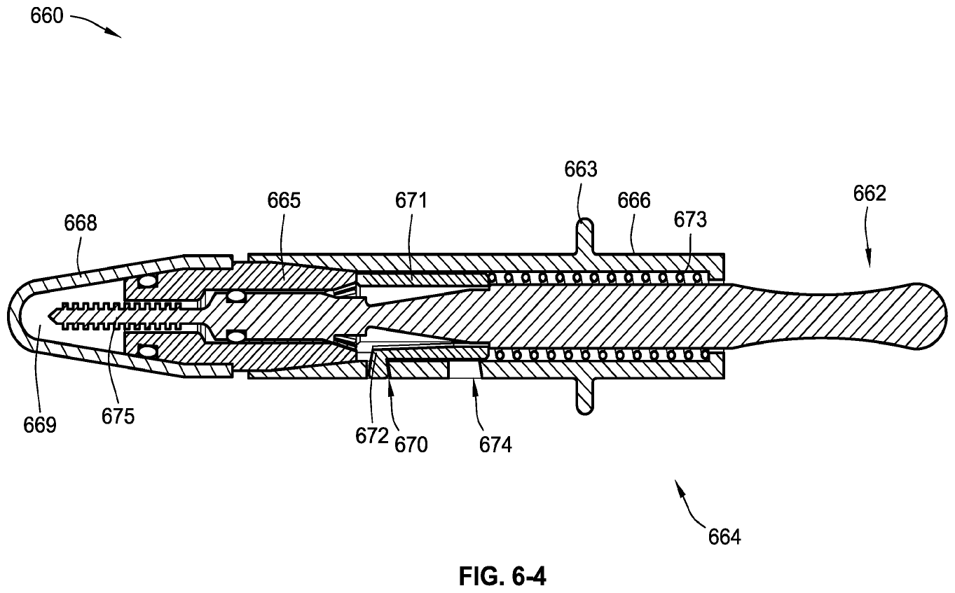

FIG. 6-1 illustrates a cross-section of a testing device 600, according to some implementations of the present disclosure. The testing device 600 includes an elongated member 602 and a tube assembly 604. The tube assembly 604 includes a first chamber casing 606 where at least a portion of the first chamber casing 606 defines a first chamber 607 and a second chamber casing 608 defining a second chamber 609. In FIG. 6, the first chamber casing 606 is a contiguous part that provides functionality similar to the combined parts of the first chamber casing 208 (FIG. 4) and the first cylindrical member 202 (FIG. 4). The tube assembly 604 includes a key opening 612 and a seal 616 similar to the key opening 212 (FIG. 4) and the seal 402 (FIG. 4). The testing device 600 is shown in the first configuration since a tip end 614 of the elongated member 602 is positioned within the first chamber 607 and has not punctured the seal 616. The tube assembly 604 can include a cap 610. The cap 610 can be removed prior to inserting the elongated member 602, or, in some implementations, the cap 610 includes a membrane that can be punctured by the elongated member 602 when inserted into the tube assembly 604.

FIGS. 6-2, 6-3, and 6-4 illustrate cross-sections of testing devices 620, 640, and 660, respectively, according to some implementations of the present disclosure. The testing devices 620 and 640 are shown in a first configuration, with a tip end 634 (654) of an elongated member 622 (642) positioned within a first chamber 627 (647) of the testing device 620 (640). A second chamber 629 (649) of the testing device 620 (640) is still sealed and separated from the first chamber 627 (647). The first chamber 627 (647) is defined by a first chamber casing 625 (645). The testing device 660 is shown in a second configuration, with a tip end 675 of an elongated member 662 positioned within a second chamber 669 of the testing device 660.

The testing device 100 of FIG. 4 has the spring retainer 206 positioned such that the spring 204 compresses only a small portion of the spring retainer 206 at one end portion thereof when in the locked position. In FIGS. 6-2 to 6-4, the testing devices 620, 640, and 660 have spring retainers 631, 651, and 671 positioned such that springs 633, 653, and 673 compress substantially the entirety of the spring retainers 631, 651, and 671, respectively. When in the locked position, the spring retainers 631, 651, and 671 are compressed by the springs 633, 653, and 673 since protrusions 632, 652, and 672 prevent the spring retainers 631, 651, and 671 from moving while the force of the springs 633, 653, and 673 is transmitted through the entirety or substantially the entirety of the spring retainers 631, 651, and 671.

Compared to the testing devices 620 and 640, the testing device 660 is shown in the second configuration. The testing device 660 includes a first key opening 674 and a second key opening 670. The first key opening 674 is similar to the key opening 212 (FIG. 4) and allows the protrusion 672 to prevent the spring retainer 671 from moving in a direction toward a second chamber 669 of the testing device 660 when in the first configuration. The second key opening 670 is optional and provided as a secondary alternative means to prevent the spring retainer 671 from further moving toward the second chamber 669 when in the second configuration.

FIG. 6-4 depicts the testing device 660 in the second configuration with multiple safeguards for preventing the elongated member 662 from further moving when the tip end 675 is situated in the second chamber 669. For example, a shape of a first chamber casing 665 interfacing with a shape of the elongated member 662 can prevent further motion, and the spring retainer 671 abutting against the first chamber casing 665 can prevent further motion, etc.

Tube assemblies 624, 644, and 664 of the testing devices 620, 640, 660, respectively, have first cylindrical members 626, 646, and 666 with flanges 623, 643, and 663. The flanges 623, 643, and 663 can help the tube assemblies 624, 644, and 664 situate vertically in holders, with the flanges 623, 643, and 663 being contact points between the tube assemblies 624, 644 and 664 and the holders. Furthermore, second chamber casings 628, 648, and 668 have different shapes and are thinner when compared to, for example, the second chamber casing 210 of FIG. 4. The tube assemblies 620 and 660 can be preferred for optics readouts, and the tube assembly 640 can be used for lab standard ‘650 μl’ tubes.

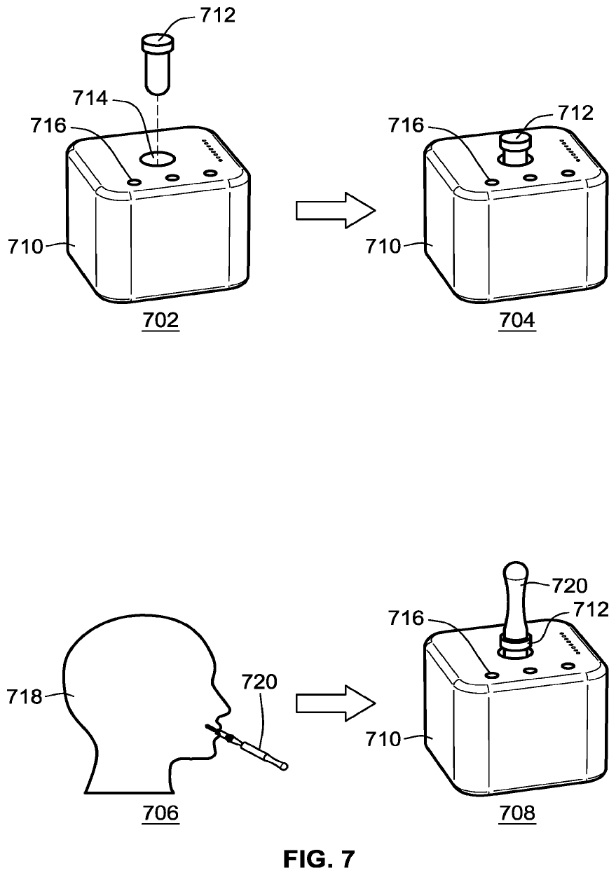

FIG. 7 illustrates a process for conducting a test, according to some implementations of the present disclosure. Step 702 illustrates an electronic device 710 having visual indicators and/or buttons 716 and a receptacle 714 for receiving a tube assembly 712. The tube assembly 712 can be similar to or the same as the tube assembly 104 of FIG. 1. Step 704 illustrates the tube assembly 712 inserted in the electronic device 710. Step 706 illustrates a subject 718 having a swab 720 collecting samples. The swab 720 is similar to or the same as the elongated member 102 of FIG. 1. Step 708 illustrates the swab 720 being inserted in the tube assembly 712 to conduct the test on the collected samples. In some implementations, the samples are biological samples obtained from a subject, e.g., human subject, in need of testing. A biological sample may be a biofluid, such as saliva, nasal fluid, mucus. The biological samples may comprise cells or genetic material (e.g., nucleic acids) from the subject, bacteria, viruses, fungi, or a combination thereof.

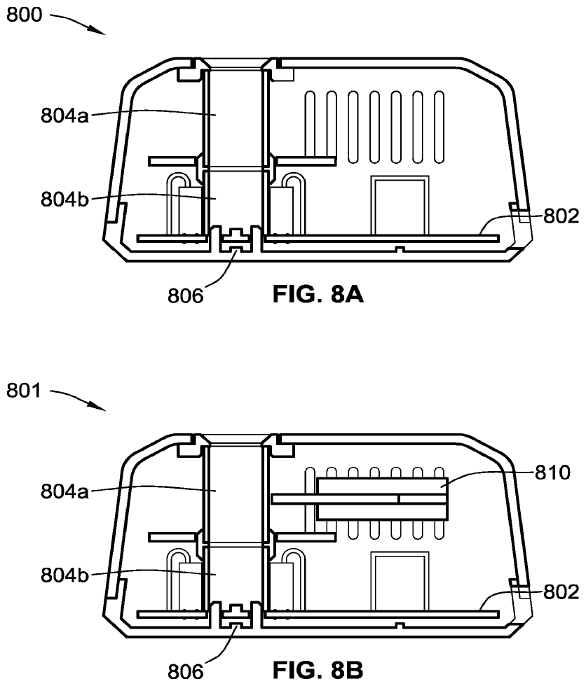

FIG. 8A illustrates a cross-section of a first electronic device 800, according to some implementations of the present disclosure. The first electronic device 800 can include a microprocessor 802, one or more heaters (e.g., a 95-degree Celsius heater, a 60-degree Celsius heater, etc.), and a fluorimeter 806 (e.g., a 3-color fluorimeter). The one or more heaters can include a first heater 804a, a second heater 804b, or additional heaters. The microprocessor 802 is configured to interpret and provide visual indicators based on the results provided by the fluorimeter 806. In some implementations, the first electronic device 800 can be engineered for a two-chambered tube assembly (e.g., the tube assembly 104 of FIG. 4). The first chamber (e.g., the first chamber 407 of FIG. 4) can be situated such that the first heater 804a of the first electronic device 800 maintains the first chamber at a first temperature (e.g., 95 degrees Celsius) and the second heater 804b of the first electronic device 800 maintains the second chamber (e.g., the second chamber 409 of FIG. 4) at a second temperature (e.g., 60 degrees Celsius).

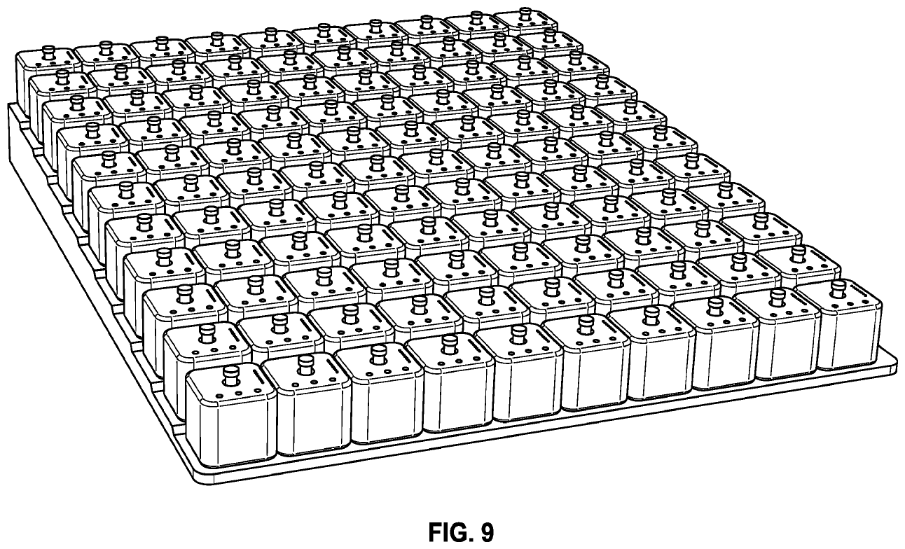

FIG. 8B illustrates a cross-section of a second electronic device 801, according to some implementations of the present disclosure. The second electronic device 801 is similar to the first electronic device 800, but the second electronic device 801 includes a solenoid 810 that can be used to dislodge a protrusion (e.g., the protrusion 316 of FIG. 4) to cause an elongated member (e.g., the elongated member 102 of FIG. 4) to advance and puncture a membrane separating two chambers of a test assembly. The first electronic device 800 (FIG. 8A) can be used with a tube assembly that requires an operator to manually advance the elongated member, while the second electronic device 801 (FIG. 8B) can be used with a tube assembly (e.g., the tube assembly 104 of FIG. 4) that provides for dislodgment of a spring retainer by a solenoid. The microprocessor 802 can be used to time the solenoid 810 in the second electronic device 801 such that a more precise timing of when to advance the elongated member is met. FIG. 9 illustrates a modular assembly of electronic devices, according to some implementations of the present disclosure. In one example, for mass testing, electronic devices (e.g., the electronic devices 800 or 801) can be arranged as depicted in FIG. 9.

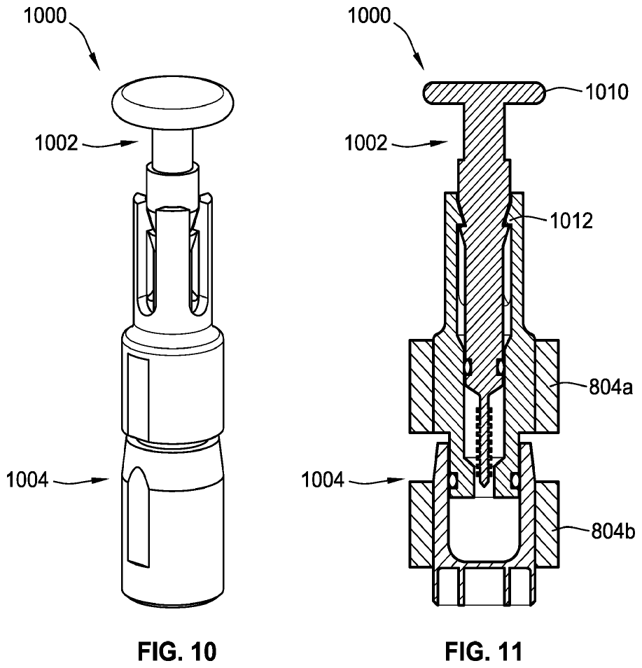

FIG. 10 illustrates a testing device 1000, according to some implementations of the present disclosure. The testing device 1000 includes an elongated member 1002 and a tube assembly 1004. FIG. 11 illustrates a cross-section of the testing device 1000, according to some implementations of the present disclosure. The elongated member 1002 includes a push button 1010 that facilitates an operator or a user to push the elongated member 1002 into the tube assembly 1004. The elongated member 1002 further includes multiple one-way detents that can clip to the tube assembly 1004 at various points 1012 as the elongated member 1002 is pushed into the tube assembly 1004. In some implementations, a click sound is produced when the elongated member 1002 is pushed into the tube assembly 1004. The click sounds can provide a human-perceivable indication to an operator that the elongated member 1002 is positioned in a first chamber of the tube assembly 1004, a second chamber of the tube assembly 1004, a third chamber of the tube assembly 1004, and so on. The human-perceivable indication allows the user to know how far to push the elongated member 1002 as reactions progress. In some implementations, the elongated member 1002 is shaped such that once a detent is reached, more force is required to push the elongated member 1002 to reach the next detent. This force feedback can also be used to provide an operator or a user an indication of how far the elongated member 1002 has been pushed into the tube assembly 1004. The manually-pushed testing device 1000 can be used with the electronic device 800 of FIG. 8A, while the testing device 100 of FIG. 4 can be used with the electronic device 801 of FIG. 8B. FIG. 11 also shows how the first heater 804a and the second heater 804b will engage with the tube assembly 1004.

Although FIGS. 10 and 11 are described in the context of a human push, other pushing mechanisms can be used. For example, force exerted from an external mechanism such as a spring can be used to push the elongated member 1002 into the tube assembly 1004. In some implementations, the external mechanism can include a geared motor, server, pneumatic piston, etc.

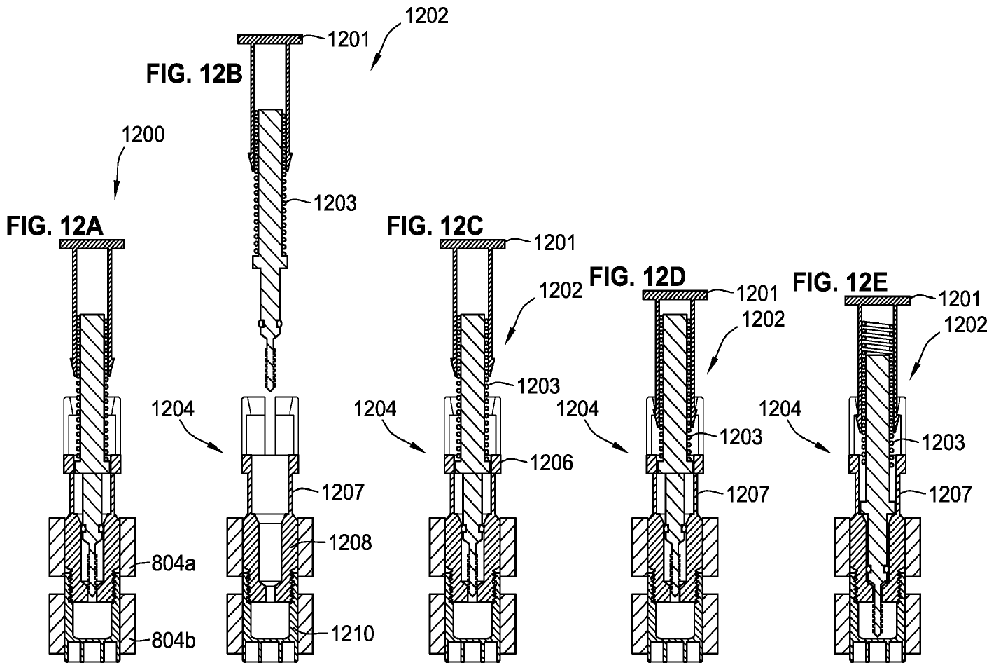

FIG. 12A illustrates a cross-section of a testing device 1200, according to some implementations of the present disclosure. FIGS. 12B-12E provide different positions of the testing device 1200. The testing device 1200 includes a tube assembly 1204 and an elongated member 1202. The elongated member 1202 includes a push button 1201 and a spring 1203. The tube assembly 1204 includes a first chamber casing 1208 that defines a first chamber and a second chamber casing 1210 that defines a second chamber. The first chamber casing 1208 also includes a thin portion 1207 that can be deformed by a solenoid to trigger. In FIG. 12B, the tube assembly 1204 and the elongated member 1202 are separated. In FIG. 12C, the tube assembly 1204 receives the elongated member 1202 until the elongated member 1202 is caught at a retainer portion 1206 of the first chamber casing 1208. The retainer portion 1206 prevents the elongated member 1202 from proceeding beyond the first chamber.

In FIG. 12D, any further downward pushes on the pushbutton 1201 results in compressing the spring 1203 while the elongated member 1202 still remains within the first chamber. The pushbutton 1201 is caught in a detent of the tube assembly 1204 such that the compressed spring 1203 stays compressed. In FIG. 12E, a solenoid can be used to deform the thin portion 1207 of the first chamber casing 1208 such that a pinching effect causes a flaring of the retainer portion 1206. Such flaring allows the spring 1203 to advance the elongated member 1202 into the second chamber as depicted in FIG. 12 E. Comparing FIGS. 12D and 12E, the spring 1203 is compressed in FIG. 12D and decompressed in FIG. 12E.

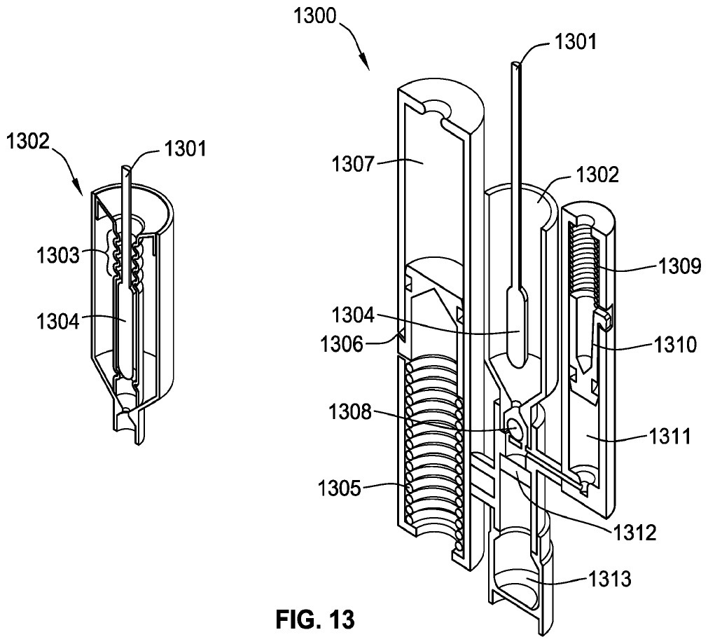

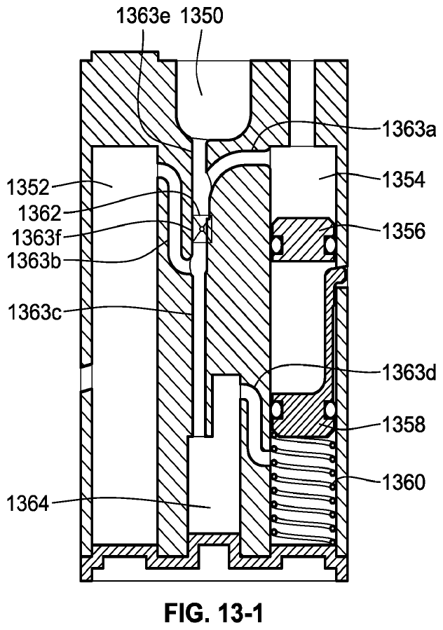

FIG. 13 illustrates a cross-section of a testing device 1300, according to some implementations of the present disclosure. The testing device 1300 includes a swab 1301 that is inserted in a sample collection and extraction chamber 1302. The swab 1301 includes a tip 1304 that contains a sample. The sample and collection chamber 1302 may contain ‘washboard’ geometry 1303 so that when the tip 1304 of the swab 1301 is inserted in the sample and collection chamber 1302, the washboard geometry 1303 can deform and massage the tip 1304 to help release the sample into extraction buffer present in the sample and collection chamber 1302. The washboard geometry 1303 relieves the user of further manipulation of the swab 1301 for optimal sample extraction. The extraction buffer present in the sample and collection chamber 1302 can include a nucleic acid extraction buffer. The nucleic acid extraction buffer can facilitate a chemical reaction resulting in nucleic acid extraction from the sample on the tip 1304 of the swab 1301.

The testing device 1300 is similar to the testing device 100 (FIG. 4) in that a spring and a spring retainer are provided in a hollow tubular member. The testing device 1300 includes a first spring 1305 provided in a first hollow tubular member 1307, shown as being compressed in FIG. 13 by a first spring retainer 1306. The first spring retainer 1306 operates in the first hollow tubular member 1307 in a similar manner as described above in FIG. 4 with respect to the first cylindrical member 202. When the first spring retainer 1306 is released, the first spring 1305 decompresses, pushing the first spring retainer 1306 and creating a vacuum in the chamber, within the first hollow tubular member 1307, where the first spring 1305 resides. Creating the vacuum within the chamber causes a pulling on the sample and extraction buffer mixture in the sample and collection chamber 1302. That is, higher pressure (e.g., atmospheric pressure) within the sample and collection chamber 1302 pushes the sample and extraction buffer mixture due to the created vacuum. The sample and extraction buffer mixture flows from the sample and collection chamber 1302 through a flexible ball 1308 acting as a check valve. The sample and extraction buffer mixture further flows through a frit (and/or filter) 1312. Although described in context of creating a vacuum, the release of the first spring 1305 can generate vacuum, or in some implementations, a positive pressure within the hollow tube member 1307.

The frit (and/or filter) 1312 can hold silica purification beads for filtering particles from the sample and extraction buffer mixture, as the mixture is guided by the generated vacuum through the frit (and/or filter) 1312. The testing device 1300 further includes a second tubular member 1311 with a second spring 1309 and a second spring retainer 1310. The second spring 1309 and the second spring retainer 1310 facilitate pushing (or injecting) fluid present in the second tubular member 1311 through silica beads provided in the frit (and/or filter) 1312. The flexible ball 1308 serves as a check-valve, preventing the pushed (or injected) fluid from entering the sample and collection chamber 1302. A post-clean/concentrate reaction chamber 1313 is provided in the testing device 1300 to collect fluids.

The components in FIG. 13 may be connected in different orientations, sizes, and shapes, with flow in different directions. FIG. 13 shows that a spring/solenoid system can be used to automate flow through and elution from silica beads or other filter, in order to clean and/or concentrate a sample such as saliva or nasal fluid.

FIG. 13-1 illustrates an aspect of the testing device 1300 of FIG. 13. A first chamber 1350 includes a buffer that soaks a swab (e.g., the swab 1301 of FIG. 13). The buffer is released from the first chamber 1350 into a waste chamber 1352 via capillaries 1363b, 1363e, 1363f. En route to the waste chamber 1352, the buffer passes through silica at position 1362. The silica filters particulates of interest as the buffer passes through. A spring 1360 is then released by triggering a spring retainer 1358 that pushes a stopper 1356. The spring retainer 1358 can be released, for example, by a solenoid, according to some implementations of the present disclosure. The stopper 1356 pushes fluid contained in the chamber 1354. In some implementations, the fluid is amplification buffer, and stopper 1356 pushes the amplification buffer from the chamber 1354 into a lower chamber 1364 via capillaries 1363a, 1363c. The amplification buffer also passes through the position 1362 en route to the lower chamber 1364, thus is also filtered in the process. The diameter of capillaries 1363a-f are small such that fluid movement is guided through negative and positive pressures created in either the waste chamber 1352 or the chamber 1354, as gravity alone would not be expected to move fluids easily through the narrow capillaries 1363a-f.

In FIG. 13-1, in some implementations, the waste chamber 1352 can include a spring and spring retainer arrangement to generate negative pressure for guiding the buffer through the capillaries 1363e, 1363f, and 1363b. Furthermore, the spring 1360, when released, can generate positive pressure that pushes amplification buffer from the chamber 1354 through the capillaries 1363a, 1363f, and into lower chamber 1364. The spring 1360, when released, also creates a negative pressure through the capillary 1363d to assist the positive pressure. Using FIG. 13, as a contrast, the first hollow tube member 1307 can serve as a waste chamber with the first spring 1305 used to create a negative pressure to draw the sample and extraction buffer into the first hollow tube member 1307. The second spring 1309 can be used to generate positive pressure in the second hollow tube member 1311 to push amplification buffer to the post clean/concentrate reaction chamber 1313.

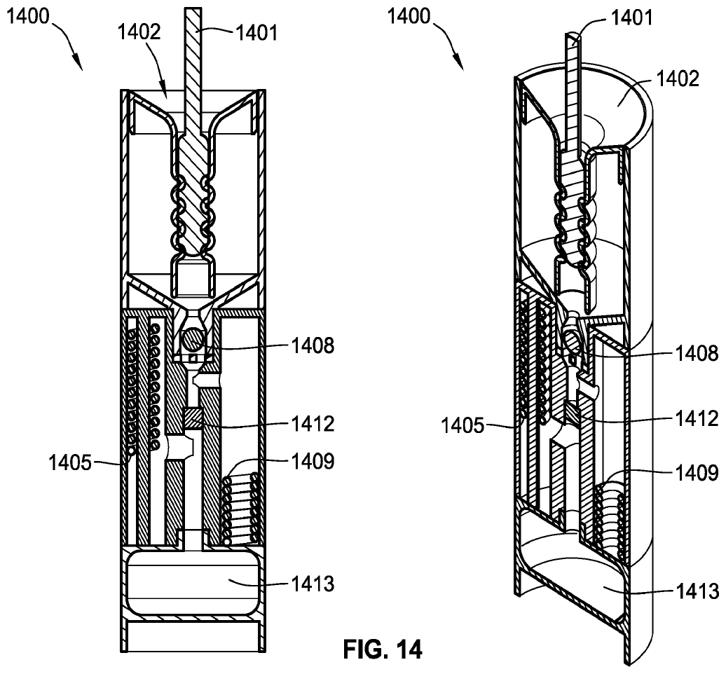

FIG. 14 illustrates a cross-section of a testing device 1400, according to some implementations of the present disclosure. The testing device 1400 operates similar to the testing device 1300 of FIG. 13. A swab 1401 is inserted in a sample collection and extraction chamber 1402 that may have a washboard geometry for automating swab massage. A first triggered spring 1405 can be used to pull vacuum on the sample through silica beads included in a frit and/or filter 1412. The frit and/or filter 1412 is provided to hold the silica purification beads. A second spring 1409 can be used to push elution/reaction buffer through the beads in the frit and/or filter 1412 and into an amplification chamber 1413. A flexible ball 1408 can be provided to operate as a check-valve. The amplification chamber 1413 serves as the post-clean concentrate reaction chamber. The testing device 1400 of FIG. 14 is contained in one cylindrical design when compared to the testing device of FIG. 13.

Components may be connected in different orientations, sizes, and shapes, with flow in different directions. Similar to FIG. 13, FIG. 14 shows that a spring/solenoid system can be used to automate flow through and elution from silica beads or other filter, in order to clean and/or concentrate a sample, such as saliva or nasal fluid.

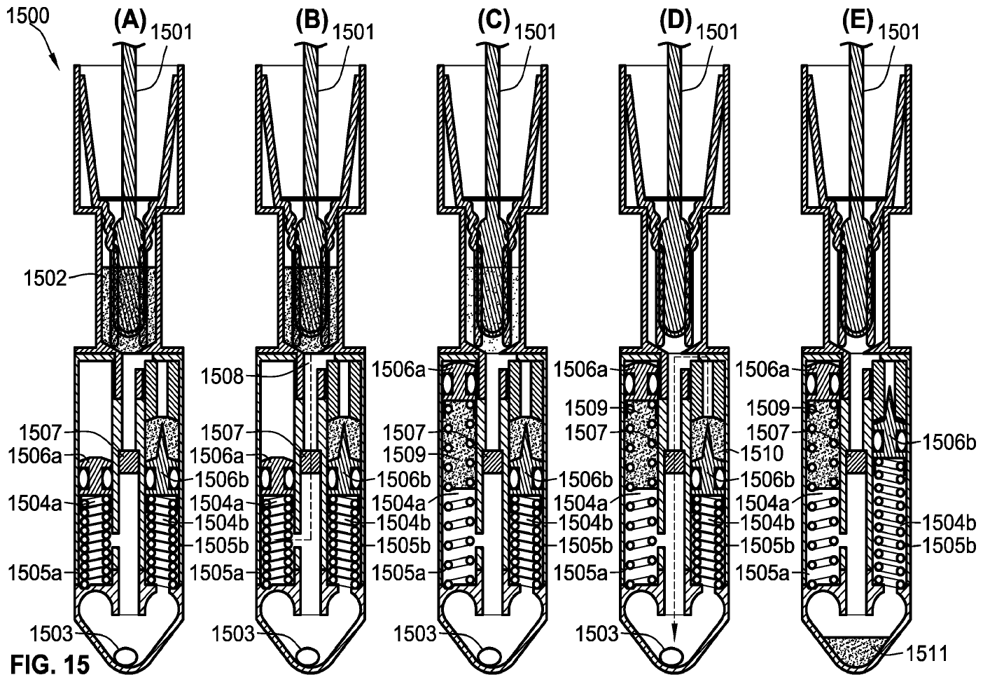

FIG. 15 illustrates steps for using a testing device 1500, according to some implementations of the present disclosure. The testing device 1500 is similar to and operates similar to the testing device 1400 of FIG. 14. Inset (A) of FIG. 15 illustrates a first step where a swab is eluted, inset (B) of FIG. 15 illustrates a second step where a first spring is activated, inset (C) of FIG. 15 illustrates a third step where a sample is applied, inset (D) of FIG. 15 illustrates a fourth step where a second spring is activated, and inset (E) of FIG. 15 illustrates a fifth step where a sample is eluted.

In inset (A) of FIG. 15, a swab 1501 is inserted in an extraction buffer 1502 in a first chamber of the testing device 1500. A lyophilized pellet 1503 is provided at a second chamber of the testing device 1500 as shown. The swab 1501 is soaked in the extraction buffer 1502 for a predetermined amount of time. The testing device 1500 includes multiple spring chambers. In FIG. 15, the testing device 1500 includes two spring chambers (a first spring chamber 1504a with a first spring 1505a and a second spring chamber 1504b with a second spring 1505b). The spring chambers 1504a and 1504b include spring retainers 1506a and 1506b, respectively, that keep the springs 1505a,b of the spring chambers 1504a,b in a pre-compressed state. The spring retainers 1506a,b are similar to or the same as spring retainers discussed above in connection with FIG. 4. A filter 1507 is also included in the testing device 1500.

In inset (B) of FIG. 15, the first spring 1505a is activated. The first spring 1505a is activated in a similar manner as discussed above in connection with releasing spring retainers, according to some implementations of the present disclosure. When activated, the first spring 1505a causes a vacuum pressure that draws the fluid (e.g., the extraction buffer 1502) from the first chamber of the testing device 1500 into the first spring chamber 1504a. The path for fluid flow is denoted with arrow 1508, and the first spring 1505a when released is as shown in inset (C) of FIG. 15. The fluid drawn into the first spring chamber 1504a passes through the filter 1507 such that particulates of interest from the swab 1501 are retained, thus the resultant fluid 1509 in the first spring chamber 1504a is waste fluid.

In inset (D) of FIG. 15, the second spring 1505b is activated. The second spring 1505b is activated in a similar manner as discussed above in connection with releasing spring retainers, according to some implementations of the present disclosure. When activated, the second spring 1505b pushes a second fluid 1510 from the second spring chamber 1504b into the second chamber with the lyophilized pellet 1503. The second fluid 1510 can be an elution or amplification buffer. Inset (E) of FIG. 15 shows position of the first and second fluids after both springs 1505a,b are activated. The mixture 1511 includes the lyophilized pellet 1503.

In FIGS. 13, 14, and 15, although described as an amplification buffer, the pushed fluid can be an elution buffer. The amplification buffer can be nucleic acid amplification reagents, including isothermal nucleic acid amplification reagents. The nucleic acid amplification reagents can include polymerase chain reaction (PCR) reagents, recombinase polymerase amplification (RPA) reagents, loop-mediated isothermal amplification (LAMP) reagents, rolling circle amplification (RCA) reagents, or strand displacement amplification (SDA) reagents.

In some implementations, nucleic acid amplification reagents are lyophilized. In some implementations, the chemical reaction occurring in the second chamber (e.g., the mixture 1511 in the second chamber of inset (E) of FIG. 15) is a nucleic acid amplification reaction. The nucleic acid amplification reaction can be a PCR, RPA, LAMP, etc. In some implementation, the second chamber contains a nucleic acid probe comprising a reporter molecule capable of producing a detectable signal. The nucleic acid probe can include a nucleotide sequence substantially complementary to an amplicon from the nucleic acid amplification. The second chamber can include an exonuclease. The exonuclease can be a double-strand specific exonuclease having 5′ to 3′ exonuclease activity.

In FIGS. 13, 14, and 15, a sample undergoes steps to obtain a processed sample in the second chamber (e.g., the lower chamber of FIG. 15). Instead of simply heating the sample within the first/middle chamber, a lab-standard ‘silica-based RNA/DNA purification’ scheme is adopted to remove impurities. In some implementations, 90% to 99% or more of non-nucleic acid impurities such as proteins, sugars, cell debris, and buffer components are removed. The final nucleic acid concentration may be lower, the same as, or higher than the original concentration, allowing for both purification and/or concentration alteration. The scheme involves: (i) mixing the sample with a larger volume buffer in the first chamber; (ii) binding nucleic acids as the sample/buffer passes through the silica pellet and into a waste chamber, drawn by a piston/spring; and (iii) driving an elution buffer using a second spring through the silica and into the lower reaction chamber.

In some implementations, a system can be configured with opposing springs in a single tube to allow for pushing and retracting fluid in the tube. In some implementations, the opposing springs allow for advancing or retracting a swab from one chamber in the tube to another. Multiple spring retainers can be used in these implementations, allowing for returning a piston or a swab to a previous position.

FIGS. 13-15 use amplification buffers and amplification reaction as an example. As used herein, “amplification” is defined as the production of additional copies of a nucleic acid sequence, i.e., for example, amplicons or amplification products. Methods of amplifying nucleic acid sequences include, but are not limited to, isothermal amplification, polymerase chain reaction (PCR) and variants of PCR such as Rapid amplification of cDNA ends (RACE), ligase chain reaction (LCR), multiplex RT-PCR, immuno-PCR, SSIPA, Real Time RT-qPCR and nanofluidic digital PCR.

Non-limiting examples of isothermal amplification include: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), and polymerase Spiral Reaction (PSR). See e.g., Yan et al., Isothermal amplified detection of DNA and RNA, March 2014, Molecular BioSystems 10(5), DOI: 10.1039/c3mb70304e, the content of which is incorporated herein by reference in its entirety.

In some implementations, samples comprise nucleic acid molecules (e.g., RNA or DNA) extracted from the subject, viruses, bacteria, etc. RNA, as used herein, can be any known type of RNA. For example, RNA can include messenger RNA, pre-mRNA, ribosomal RNA, Signal recognition particle RNA, Transfer RNA, Transfer-messenger RNA, Small nuclear RNA, Small nucleolar RNA, SmY RNA, Small Cajal body-specific RNA, Guide RNA, Ribonuclease P, Ribonuclease MRP, Y RNA, Telomerase RNA Component, Spliced Leader RNA, Antisense RNA, Cis-natural antisense transcript, CRISPR RNA, Long noncoding RNA, MicroRNA, Piwi-interacting RNA, Small interfering RNA, Short hairpin RNA, Trans-acting siRNA, Repeat associated siRNA, 7SK RNA, Enhancer RNA, Parasitic RNAs, Type, Retrotransposon, Viral genome (e.g., viral RNA), Viroid, Satellite RNA, or Vault RNA. DNA, used herein, can include genomic DNA, mitochondrial DNA, viral DNA, complementary DNA (cDNA), single-stranded DNA, double-stranded DNA, circular DNA, etc.

In some implementations, the viral genome is extracted from an RNA virus that is a Group III (i.e., double stranded RNA (dsRNA)) virus. In some implementations, the Group III RNA virus belongs to a viral family selected from the group consisting of Amalgaviridae, Birnaviridae, Chrysoviridae, Cystoviridae, Endornaviridae, Hypoviridae, Megabirnaviridae, Partitiviridae, Picobirnaviridae, Reoviridae (e.g., Rotavirus), Totiviridae, Quadriviridae. In some implementations, the Group III RNA virus belongs to the Genus Botybirnavirus. In some implementations, the Group III RNA virus is an unassigned species selected from the group consisting of: Botrytis porri RNA virus 1, Circulifer tenellus virus 1, Colletotrichum camelliae filamentous virus 1, Cucurbit yellows associated virus, Sclerotinia sclerotiorum debilitation-associated virus, and Spissistilus festinus virus 1.

In some implementations, the viral genome is extracted from an RNA virus that is a Group IV (i.e., positive-sense single stranded (ssRNA)) virus. In some implementations, the Group IV RNA virus belongs to a viral order selected from the group consisting of Nidovirales, Picornavirales, and Tymovirales. In some implementations, the Group IV RNA virus belongs to a viral family selected from the group consisting of Arteriviridae, Coronaviridae (e.g., Coronavirus, SARS-CoV), Mesoniviridae, Roniviridae, Dicistroviridae, Iflaviridae, Marnaviridae, Picornaviridae (e.g., Poliovirus, Rhinovirus (a common cold virus), Hepatitis A virus), Secoviridae (e.g., sub Comovirinae), Alphaflexiviridae, Betaflexiviridae, Gammaflexiviridae, Tymoviridae, Alphatetraviridae, Alvernaviridae, Astroviridae, Barnaviridae, Benyviridae, Bromoviridae, Caliciviridae (e.g., Norwalk virus), Carmotetraviridae, Closteroviridae, Flaviviridae (e.g., Yellow fever virus, West Nile virus, Hepatitis C virus, Dengue fever virus, Zika virus), Fusariviridae, Hepeviridae, Hypoviridae, Leviviridae, Luteoviridae (e.g., Barley yellow dwarf virus), Polycipiviridae, Narnaviridae, Nodaviridae, Permutotetraviridae, Potyviridae, Sarthroviridae, Statovirus, Togaviridae (e.g., Rubella virus, Ross River virus, Sindbis virus, Chikungunya virus), Tombusviridae, and Virgaviridae. In some implementations, the Group IV RNA virus belongs to a viral genus selected from the group consisting of Bacillariornavirus, Dicipivirus, Labyrnavirus, Sequiviridae, Blunervirus, Cilevirus, Higrevirus, Idaeovirus, Negevirus, Ourmiavirus, Polemovirus, Sinaivirus, and Sobemovirus. In some implementations, the Group IV RNA virus is an unassigned species selected from the group consisting of Acyrthosiphon pisum virus, Bastrovirus, Blackford virus, Blueberry necrotic ring blotch virus, Cadicistrovirus, Chara australis virus, Extra small virus, Goji berry chlorosis virus, Hepelivirus, Jingmen tick virus, Le Blanc virus, Nedicistrovirus, Nesidiocoris tenuis virus 1, Niflavirus, Nylanderia fulva virus 1, Orsay virus, Osedax japonicus RNA virus 1, Picalivirus, Plasmopara halstedii virus, Rosellinia necatrix fusarivirus 1, Santeuil virus, Secalivirus, Solenopsis invicta virus 3, Wuhan large pig roundworm virus. In some implementations, the Group IV RNA virus is a satellite virus selected from the group consisting of: Family Sarthroviridae, Genus Albetovirus, Genus Aumaivirus, Genus Papanivirus, Genus Virtovirus, and Chronic bee paralysis virus.

In some implementations, the viral genome is extracted from an RNA virus that is a Group V (i.e., negative-sense ssRNA) virus. In some implementations, the Group V RNA virus belongs to a viral phylum or subphylum selected from the group consisting of: Negarnaviricota, Haploviricotina, and Polyploviricotina. In some implementations, the Group V RNA virus belongs to a viral class selected from the group consisting of: Chunqiuviricetes, Ellioviricetes, Insthoviricetes, Milneviricetes, Monjiviricetes, and Yunchangviricetes. In some implementations, the Group V RNA virus belongs to a viral order selected from the group consisting of: Articulavirales, Bunyavirales, Goujianvirales, Jingchuvirales, Mononegavirales, Muvirales, and Serpentovirales. In some implementations, the Group V RNA virus belongs to a viral family selected from the group consisting of: Amnoonviridae (e.g., Taastrup virus), Arenaviridae (e.g., Lassa virus), Aspiviridae, Bornaviridae (e.g., Borna disease virus), Chuviridae, Cruliviridae, Feraviridae, Filoviridae (e.g., Ebola virus, Marburg virus), Fimoviridae, Hantaviridae, Jonviridae, Mymonaviridae, Nairoviridae, Nyamiviridae, Orthomyxoviridae (e.g., Influenza viruses), Paramyxoviridae (e.g., Measles virus, Mumps virus, Nipah virus, Hendra virus, and NDV), Peribunyaviridae, Phasmaviridae, Phenuiviridae, Pneumoviridae (e.g., RSV and Metapneumovirus), Qinviridae, Rhabdoviridae (e.g., Rabies virus), Sunviridae, Tospoviridae, and Yueviridae. In some implementations, the Group V RNA virus belongs to a viral genus selected from the group consisting of: Anphevirus, Arlivirus, Chengtivirus, Crustavirus, Tilapineviridae, Wastrivirus, and Deltavirus (e.g., Hepatitis D virus).

In some implementations, the viral genome is extracted from an RNA virus that is a Group VI RNA virus, which comprise a virally encoded reverse transcriptase. In some implementations, the Group VI RNA virus belongs to the viral order Ortervirales. In some implementations, the Group VI RNA virus belongs to a viral family or subfamily selected from the group consisting of: Belpaoviridae, Caulimoviridae, Metaviridae, Pseudoviridae, Retroviridae (e.g., Retroviruses, e.g. HIV), Orthoretrovirinae, and Spumaretrovirinae. In some implementations, the Group VI RNA virus belongs to a viral genus selected from the group consisting of: Alpharetrovirus (e.g., Avian leukosis virus; Rous sarcoma virus), Betaretrovirus (e.g., Mouse mammary tumour virus), Bovispumavirus (e.g., Bovine foamy virus), Deltaretrovirus (e.g., Bovine leukemia virus; Human T-lymphotropic virus), Epsilonretrovirus (e.g., Walleye dermal sarcoma virus), Equispumavirus (e.g., Equine foamy virus), Felispumavirus (e.g., Feline foamy virus), Gammaretrovirus (e.g., Murine leukemia virus; Feline leukemia virus), Lentivirus (e.g., Human immunodeficiency virus 1; Simian immunodeficiency virus; Feline immunodeficiency virus), Prosimiispumavirus (e.g., Brown greater galago prosimian foamy virus), and Simiispumavirus (e.g., Eastern chimpanzee simian foamy virus).

In some implementations, the viral genome is extracted from an RNA virus that is selected from influenza virus, human immunodeficiency virus (HIV), and severe acute respiratory syndrome coronavirus 2 (SARS-CoV-2). In some implementations, the RNA virus is influenza virus. In some implementations, the RNA virus is immunodeficiency virus (HIV). In some implementations, the RNA virus is severe acute respiratory syndrome coronavirus 2 (SARS-CoV-2).

In some implementations, the viral RNA is an RNA molecule produced by a virus with a DNA genome, i.e., a DNA virus. As a non-limiting example the DNA virus is a Group I (dsDNA) virus, a Group II (ssDNA) virus, or a Group VII (dsDNA-RT) virus.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

One or more elements or aspects or steps, or any portion(s) thereof, from one or more of any of claims 1-53 below can be combined with one or more elements or aspects or steps, or any portion(s) thereof, from one or more of any of the other claims 1-53 or combinations thereof, to form one or more additional implementations and/or claims of the present disclosure.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

What is claimed is:

1. A device comprising:
an elongated member; and
a tube assembly having a first end and a second end, the tube assembly configured to receive the elongated member at the second end, the tube assembly including:
a plurality of chambers, including a first chamber and a second chamber, the first chamber and the second chamber being separated by a membrane;
a spring positioned at the second end of the tube assembly;
a spring retainer configured to prevent the spring from decompressing when in a locked position and permit the spring to decompress when in an unlocked position.

2. The device of claim 1, wherein the tube assembly further includes a key opening for receiving a portion of the spring retainer when the spring retainer is in the locked position.

3. The device of claim 2, wherein a longitudinal axis of the tube assembly and an axis of the key opening are orthogonal.

4. The device of claim 2 or claim 3, wherein dislodging the portion of the spring retainer from the key opening places the spring retainer in the unlocked position.

5. The device of claim 4, wherein a shape of the spring retainer is deformed when the portion of the spring retainer is dislodged from the key opening.

6. The device of any one of claims 1 to 5, wherein the spring is configured to push the spring retainer towards the first end of the tube assembly when in the unlocked position.

7. The device of claim 6, wherein the elongated member moves towards the first end of the tube assembly as the spring retainer is pushed towards the first end of the tube assembly.

8. The device of claim 6 or claim 7, wherein the elongated member punctures the membrane separating the first chamber from the second chamber in response to the spring retainer being pushed towards the first end of the tube assembly.

9. The device of any one of claims 2 to 8, wherein the spring retainer is hollow and substantially cylindrical, having an internal radius that is less than an internal radius of the tube assembly.

10. The device of any one of claims 1 to 9, wherein the elongated member has a varying cross-sectional area along the length of the elongated member.

11. The device of any one of claims 1 to 10, wherein the spring retainer is further configured to secure the elongated member within the tube assembly when received at the second end of the tube assembly.

12. The device of any one of claims 1 to 11, wherein the elongated member includes flaps configured to prevent the elongated member from being removed from the tube assembly.

13. The device of any one of claims 1 to 12, wherein the first chamber includes a first reagent and the second chamber includes a second reagent different from the first reagent.

14. The device of any one of claims 1 to 13, wherein the elongated member is a syringe or a swab.

15. The device of any one of claims 1 to 14, wherein the spring retainer is transitioned from the locked position to the unlocked position using a solenoid.

16. The device of any one of claims 1 to 15, wherein the spring includes a compressed gas device or a mechanical spring.

17. The device of any one of claims 1 to 16, wherein the plurality of chambers are arranged in series, the plurality of chambers further including at least a third chamber separated from the first chamber and the second chamber.

18. An assembly including a plurality of devices according to any one of claims 1 to 17 for processing a plurality of samples, the plurality of devices arranged in an array and configured to process the plurality of samples in parallel or in serial.

19. A method for conducting chemical reactions, comprising:
inserting a swab in a first chamber of a testing device, the first chamber containing a first fluid mixture;
decompressing a first spring positioned in a first spring chamber of the testing device, the first spring decompression causing the fluid mixture in the first chamber to flow into the first spring chamber of the testing device, the fluid mixture being filtered by silica en route to the first spring chamber; and
decompressing a second spring positioned in a second spring chamber of the testing device, the second spring decompression causing a second fluid within the second spring chamber of the testing device to be filtered by the silica en route to a second chamber.

20. The method of claim 19, wherein decompressing the first spring causes a vacuum pressure while decompressing the second spring causes a positive pressure.

21. The method of claim 19 or claim 20, wherein the first and the second springs are decompressed simultaneously.

22. The method of claim 19, wherein the first spring is decompressed before the second spring is decompressed.

23. The method of any one of claims 19 to 22, wherein the swab comprises a biological sample.

24. The method of claim 23, wherein the biological sample comprises saliva, mucus, or nasal fluid.

25. The method of any one of claims 19 to 24, wherein the first fluid mixture comprises an extraction buffer.

26. The method of claim 25, wherein the extraction buffer comprises a nucleic acid extraction buffer.

27. The method of any one of claims 19 to 26, wherein a chemical reaction occurring in the first chamber is a nucleic acid extraction.

28. The method of any one of claims 19 to 27, wherein the second fluid mixture comprises an elution buffer or amplification buffer.

29. The method of any one of claims 19 to 28, wherein the second chamber contains nucleic acid amplification reagents.

30. The method of claim 29, wherein the nucleic acid amplification reagents are isothermal nucleic acid amplification reagents.

31. The method of claim 29 or claim 30, wherein the nucleic acid amplification reagents comprise polymerase chain reaction (PCR) reagents, recombinase polymerase amplification (RPA) reagents, loop-mediated isothermal amplification (LAMP) reagents, rolling circle amplification (RCA) reagents, or strand displacement amplification (SDA) reagents.

32. The method of any one of claims 29 to 31, wherein the nucleic acid amplification reagents are lyophilized.

33. The method of any one of claims 19 to 32, wherein a chemical reaction occurring in the second chamber is a nucleic acid amplification reaction.

34. The method of claim 33, wherein the nucleic acid amplification reaction is polymerase chain reaction (PCR), recombinase polymerase amplification (RPA), or loop-mediated isothermal amplification (LAMP).

35. The method of any one of claims 19 to 34, wherein the second chamber contains a nucleic acid probe comprising a reporter molecule capable of producing a detectable signal, wherein the nucleic acid probe comprises a nucleotide sequence substantially complementary to an amplicon from the nucleic acid amplification.

36. The method of any one of claims 19 to 35, wherein the second chamber contains an exonuclease.

37. The method of claim 36, wherein the exonuclease is a double-strand specific exonuclease having 5′ to 3′ exonuclease activity.

38. A method for conducting chemical reactions, comprising:
inserting one end of an elongated member into a second end of a tube assembly such that the one end of the elongated member extends into a first chamber of the tube assembly;
decompressing a spring positioned at the second end of the tube assembly by unlocking a spring retainer of the tube assembly; and
puncturing a membrane separating the first chamber of the tube assembly from a second chamber of the tube assembly such that the one end of the elongated member extends into the second chamber of the tube assembly.

39. The method of claim 38, wherein the one end of the elongated member comprises a biological sample.

40. The method of claim 39, wherein the biological sample comprises saliva or nasal fluid.

41. The method of any one of claims 38 to 40, wherein the first chamber of the tube assembly includes a first fluid mixture interacting with the one end of the elongated member, the first fluid mixture comprising an extraction buffer.

42. The method of claim 41, wherein the extraction buffer comprises a nucleic acid extraction buffer.

43. The method of any one of claims 38 to 42, wherein a chemical reaction occurring in the first chamber of the tube assembly is a nucleic acid extraction.

44. The method of any one of claims 38 to 43, wherein the second chamber of the tube assembly includes a second fluid mixture, the second fluid mixture comprising an elution buffer or amplification buffer.

45. The method of any one of claims 38 to 44, wherein the second chamber of the tube assembly contains nucleic acid amplification reagents.

46. The method of claim 45, wherein the nucleic acid amplification reagents are isothermal nucleic acid amplification reagents.

47. The method of claim 45 or claim 46, wherein the nucleic acid amplification reagents comprise polymerase chain reaction (PCR) reagents, recombinase polymerase amplification (RPA) reagents, loop-mediated isothermal amplification (LAMP) reagents, rolling circle amplification (RCA) reagents, or strand displacement amplification (SDA) reagents.

48. The method of any one of claims 45 to 47, wherein the nucleic acid amplification reagents are lyophilized.

49. The method of any one of claims 38 to 48, wherein a chemical reaction occurring in the second chamber is a nucleic acid amplification reaction.

50. The method of claim 49, wherein the nucleic acid amplification reaction is polymerase chain reaction (PCR), recombinase polymerase amplification (RPA), or loop-mediated isothermal amplification (LAMP).

51. The method of any one of claims 38 to 50, wherein the second chamber contains a nucleic acid probe comprising a reporter molecule capable of producing a detectable signal, wherein the nucleic acid probe comprises a nucleotide sequence substantially complementary to an amplicon from the nucleic acid amplification.

52. The method of any one of claims 38 to 51, wherein the second chamber contains an exonuclease.

53. The method of claim 52, wherein the exonuclease is a double-strand specific exonuclease having 5′ to 3′ exonuclease activity.
